(19) United States
(12) Patent Application Publication
(10) Pub. No.: US 2007/0149850 A1
(43) Pub. Date: Jun. 28, 2007

(54) Endoscope endcap attachment tool

(76) Inventors: James T. Spivey, Loveland, OH (US); David Stefanchik, Morrow, OH (US); Rich D. Applegate, Florence, KY (US)

Correspondence Address: Thompson Hine LLP;2000 Courthouse Plaza N.E., 10 West Second Street, Dayton, OH, 45402-1758, US

(21) Appl. No.: 11/315,887

(22) Filed: Dec. 22, 2005

(51) Int. Cl. A61B 1/04

(52) U.S. Cl. 600/124; 600/127

(57) ABSTRACT

A tool and method is provided for attaching an endcap to a medical instrument such as an endoscope tip, wherein the endcap includes an elastically deformable, endcap opening having an unexpanded diameter that is smaller than the diameter of the instrument tip and an expanded diameter that is greater than the diameter of the instrument tip. The tool has a first component adapted to hold the endcap and to receive the instrument tip and a second component operatively associated with the first component and movable with respect to the first component, wherein the second component is adapted to apply a force to the first component. The endcap opening may be elastically deformed to have the expanded diameter prior to positioning the endcap at a desired longitudinal location and angular orientation with respect to the endoscope tip.

This patent application is related to the following patent applications, which are hereby incorporated herein for reference:

U.S. Ser. No. 10/440,957 (published as US 2004/0230095), filed May 12, 2003;

U.S. Ser. No. 10/440,660 (published as US 2004/0230096), filed May 12, 2003; and

U.S. Ser. No. 10/440,956 (published US 2004/0230097), filed May 16, 2003.

### BACKGROUND

The present application relates to devices and methods for endoscopic medical procedures and, more particularly, to devices and methods for flexible endoscopic medical procedures.

There are currently many varieties of endoscopes available for performing medical procedures through small incisions or natural body orifices in the body of a patient. Some types of endoscopes are adapted only for illuminating and viewing a wound or disease site inside a body cavity. Other types of endoscopes also include working channels for introducing instruments, removing tissue and other purposes. For example, gastroenterologists use long, flexible shaft endoscopes (gastroscopes, colonoscopes, etc.) to view and access tissues inside the gastrointestinal tract via the mouth or anus. Such flexible endoscopes usually include a working channel having a diameter in the range of two to five millimeters, thereby limiting the size, shape and number of medical instruments that may be introduced into and the size of tissues removed from a patient's body.

As a consequence, many accessories, sheaths, overtubes, attachments, and other types of medical apparatuses have been developed for use with endoscopes for various purposes. For example, overtubes that cover the shaft of the endoscope have been developed to help keep the endoscope clean or to provide auxiliary endoscopic passageways or guides into the patient's body. Some of these apparatuses include an attachment device or endcap that may be releasably attached to the endoscope tip. Some endcaps may be clamped or pressed tightly onto the endoscope tip. (See U.S. Pat. application Ser. No. 10/440,957 “Medical Apparatus for Use with an Endoscope”, Stefanchik et al., filed May 16, 2003.)

Another type of endcap may be formed from an elastomeric material that is adapted to fit tightly around the periphery of the endoscope tip. It may be difficult to grip the endoscope (especially if assembled with a sheath or overtube) and to apply the appropriate force to urge the endcap over the endoscope tip. Additionally, it may be desirable to maintain a certain angular “o'clock” orientation and longitudinal placement of the endcap with respect to the endoscope tip. In the course of applying the force to urge the endcap onto the endoscope tip, the desired angular orientation and/or longitudinal position may be inadvertently lost.

Accordingly, there is a need for an improved attachment tool and method for attaching an endcap to an endoscope tip at a desired angular orientation and longitudinal position with respect to the endoscope tip.

### SUMMARY

A tool and method is provided for attaching an endcap to a tubular instrument such as an endoscope, wherein the endcap is elastically deformable and has an endcap opening having an unexpanded diameter that is smaller than the outer diameter of the instrument and an expanded diameter that is greater than the diameter of the endoscope tip. The tool has a first component adapted to hold the endcap and to receive the endoscope tip and a second component operatively associated with the first component and movable with respect to the first component, wherein the second component is adapted to apply a force to the first component. The endcap opening may be elastically deformed to have the expanded diameter prior to positioning the endcap at a desired longitudinal location and angular orientation with respect to the endoscope tip.

The method includes providing the tool described in the previous paragraph, inserting the first component into the opening of the endcap, applying a force to the first component by moving the second component with respect to the first component such that the opening of the endcap has the expanded diameter, and positioning the endcap at a desired longitudinal location and angular orientation with respect to the endoscope tip.

Other aspects, variations, and embodiments of the attachment tool and method will become apparent from the following description, the accompanying drawings, and the appended claims.

### BRIEF DESCRIPTION OF THE DRAWINGS

FIG. 1 is an isometric view of a first embodiment of an attachment tool for attaching an endcap to an endoscope tip;

FIGS. 2 through 7 are partial, sectional views that illustrate a method of using the attachment tool of FIG. 1 to attach an endcap of a medical apparatus to an endoscope tip;

FIG. 2 shows an endoscope having a medical apparatus placed thereon so that the end cap is aligned with but not attached to the endoscope tip, and a temporary carrier of the attachment tool axially aligned with the endoscope tip and the endcap;

FIG. 3 shows a insertion end of the temporary carrier positioned inside the endcap and a plunger of the attachment tool partially inserted into the temporary carrier;

FIG. 4 shows the plunger forcefully extended through the temporary carrier, thereby expanding the insertion end of the temporary carrier and the endcap;

FIG. 5 shows the plunger partially withdrawn and the endoscope tip positioned inside the insertion end of the temporary carrier and the endcap;

FIG. 6 shows the temporary carrier being separated from the end cap as a user holds a retaining component against the endcap to maintain the desired longitudinal position and angular orientation of the end cap relative to the endoscope tip;

FIG. 7 shows the temporary carrier and plunger separated from the endcap and the end cap attached to the endoscope tip;

FIG. 8 is a front view of a second embodiment of an attachment tool for attaching an endcap to an endoscope tip, showing a plunger partially inserted into a temporary carrier of the attachment tool;

FIG. 9 is a front view of the attachment tool shown in FIG. 8, showing the plunger withdrawn from, but still attached to the temporary carrier by a pair of flexible connecting elements;

FIG. 10 is a side view of the plunger shown in FIG. 9, wherein the flexible connecting elements have been omitted for clarity;

FIG. 11 is a cross-sectional view taken at line 11-11 of the plunger shown in FIG. 10;

FIG. 12 is an isometric view of a third embodiment of an attachment tool for attaching an endcap to an endoscope tip;

FIG. 13 is a top view of the attachment tool shown in FIG. 12;

FIG. 14 is a partial, isometric view of an alternate version of a temporary carrier and a retaining component;

FIG. 15 is a longitudinal section of a portion of the exchange and retaining components shown in FIG. 14 as they may be used with the plunger of FIG. 8 to position an end cap on an endoscope tip;

FIG. 16 is an end view of another alternate version of a retaining component;

FIG. 17 is a side view of the retaining component shown in FIG. 16;

FIG. 18 is an isometric view of the retaining component shown in FIG. 16; and

FIG. 19 is an isometric view of a prior art, medical apparatus including an endcap for use with an endoscope.

### DETAILED DESCRIPTION

A physician or medical assistant (“user”) may use the improved attachment tool and method described herein for attaching an endcap to an endoscope tip at a desired longitudinal position and angular orientation with respect to the endoscope tip. The endcap may include any device having an elastically deformable endcap opening and adapted for attaching tightly around the periphery of the endoscope tip (also referred to as the distal end of the endoscope). The endcap opening may have an unexpanded diameter that is smaller than the diameter of the endoscope tip, and an expanded diameter that is larger than the diameter of the endoscope tip. The user may use the attachment tool and method disclosed herein to stretch the endcap opening to the expanded diameter prior to positioning the endcap on the endoscope tip so that it is not necessary to forcibly insert the endoscope tip into the endcap opening as with previous devices.

The conventional usage of the terms “distal” and “proximal” shall be used herein. To avoid confusion, however, it should be noted that the attachment tool may be used such that the distal direction when referring to the endoscope is opposite of the distal direction when referring to the attachment tool.

FIG. 19 is an isometric view of an exemplary medical instrument 210, that may be used with the attachment tool and method described herein. A detailed description of medical apparatus 210 may be found in the earlier referenced patent applications: U.S. Ser. No. 10/440,957; U.S. Ser. No. 10/440,660; and U.S. Ser. No. 10/440,956. According to one embodiment, apparatus 210 may include an elastomeric endcap 218, a handle 212, a flexible sheath 214 extending from handle 212, and a flexible track 216 disposed on sheath 214. Handle 212 and sheath 214 may each be sized to receive an endoscope therethrough. Endcap 218 may be formed from a thermoplastic elastomer such as Santoprene brand thermoplastic elastomer and sized to fit tightly around the periphery of the endoscope tip. The durometer of the elastomer may be in a range, for example, of approximately 50-100, as measured according to ASTM D2240. Endcap 218 may include a generally cylindrical body portion, a distal face, a proximal face, and a central bore opening therethrough for receiving the endoscope tip. Endcap 218 may include numerous other features, as described in the referenced patent applications.

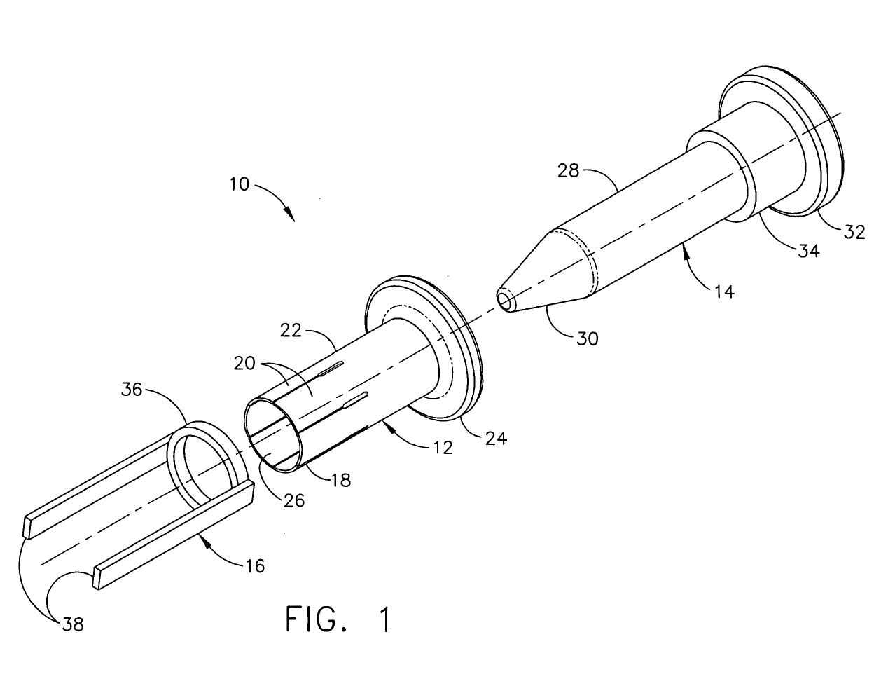

FIG. 1 is an isometric view of a first embodiment of an attachment tool, generally designated 10, for attaching an endcap (formed from an elastic material) to an endoscope. Attachment tool 10 includes a temporary carrier 12, a plunger 14 and a retaining component 16. Attachment tool 10 may be formed such as by injection molding from one or more of numerous polymers, including polycarbonate, polyetherimide and polyethylene. Attachment tool 10 may be adapted for single or multiple procedure use.

Temporary carrier 12 may include a cylindrical body 22 having a bore 26 therethrough. Body 22 may include a plurality of parallel, flexible fingers 20 extending from body 22 and arranged closely side-by-side around the periphery of an insertion end 18. FIG. 1 shows temporary carrier 12 to have eight fingers 20, although the number of fingers 20 may vary. When a radially inward force is uniformly applied to the periphery of insertion end 18 (such as when insertion end 18 is holding an expanded, elastomeric endcap), fingers 20 support each other, much like a keystone set into a stone arch holds the adjoining members in place, so that insertion end 18 maintains an expanded configuration. However, when a radially inward force is non-uniformly applied to the periphery of insertion end 18 (such as when a user pinches fingers 20 together), fingers 20 may bend radially inward so that insertion end 18 may assume a collapsed configuration.

Temporary carrier 12 may also include a handle 24 on the proximal end (opposite of the insertion end) of body 22. Handle 24, as shown in FIG. 1, may be configured as a flange on the proximal end of body 22.

Plunger 14 may include a plunger body 28 having a distal, tapered end 30 and a proximal, actuating end 32. As shown in FIG. 1, actuating end 32 may also be configured as a flange. Plunger 14 may further include a plunger stop 34 disposed near the proximal end of body 28 for limiting the insertion depth of plunger 14 into bore 26 of temporary carrier 12. Plunger body 28 may have a circular cross-sectional profile that is hollow or solid. The outer diameter of plunger body 28 may be sized to form a close, sliding fit inside of bore 26 of temporary carrier 12. A surgical lubricant such as KYJelly™ (Johnson & Johnson Corp.) may be applied to plunger body 28 to facilitate insertion into bore 26.

Retaining component 16 may include a ring 36 that may be slidingly retained over body 22 of temporary carrier 22. Retaining component 16 may also include at least one extension element 16 attached to ring 36 and extending distally as shown in FIG. 1.

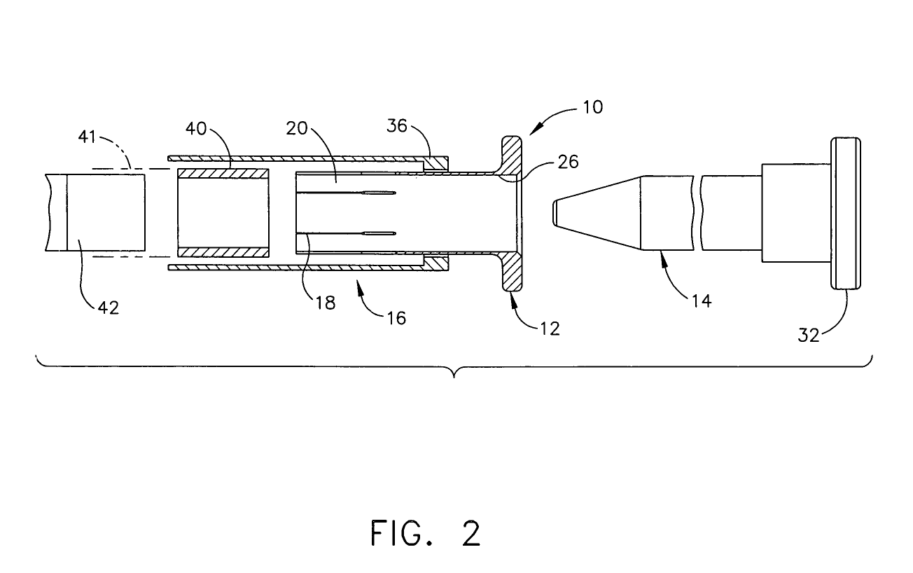

FIGS. 2 through 7 illustrate a method of using the attachment tool of FIG. 1 to attach an end cap 40 to an endoscope tip 42, such as after the endoscope has been cleaned and disinfected in the conventional manner in preparation for a medical procedure.

FIG. 2 shows endoscope tip 42 provided with a medical apparatus 41 (partially shown in phantom) placed thereon so that end cap 40 is aligned with endoscope tip 42. Temporary carrier 12 of attachment tool 10 is shown as it may be held in axial alignment with endoscope tip 42 and end cap 40. Insertion end 18 is shown in the expanded configuration.

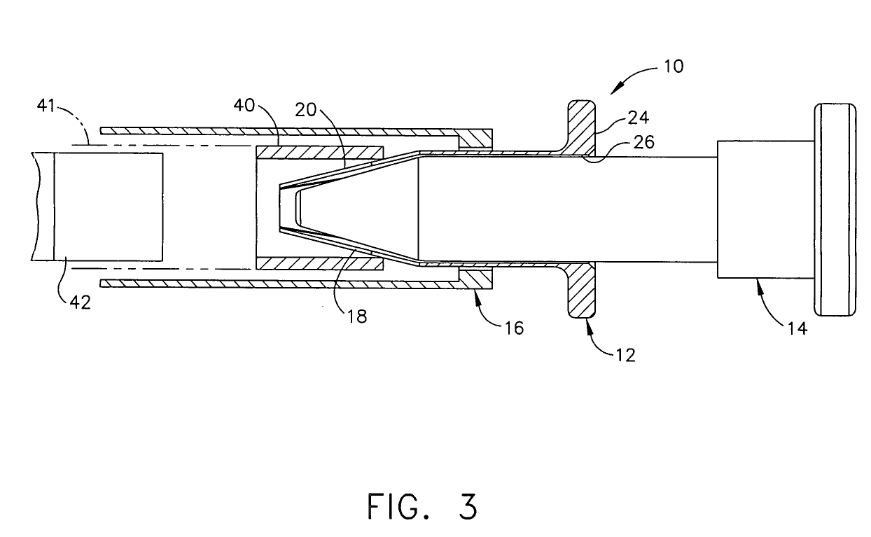

FIG. 3 shows insertion end 18 of temporary carrier 12 positioned inside end cap 40 and plunger 14 of attachment tool 10 partially inserted into bore 26 of temporary carrier 12. The user may pinch and hold fingers 20 to change insertion end 18 to the collapsed configuration shown so that insertion end 18 may be inserted into end cap 40 to a desired depth.

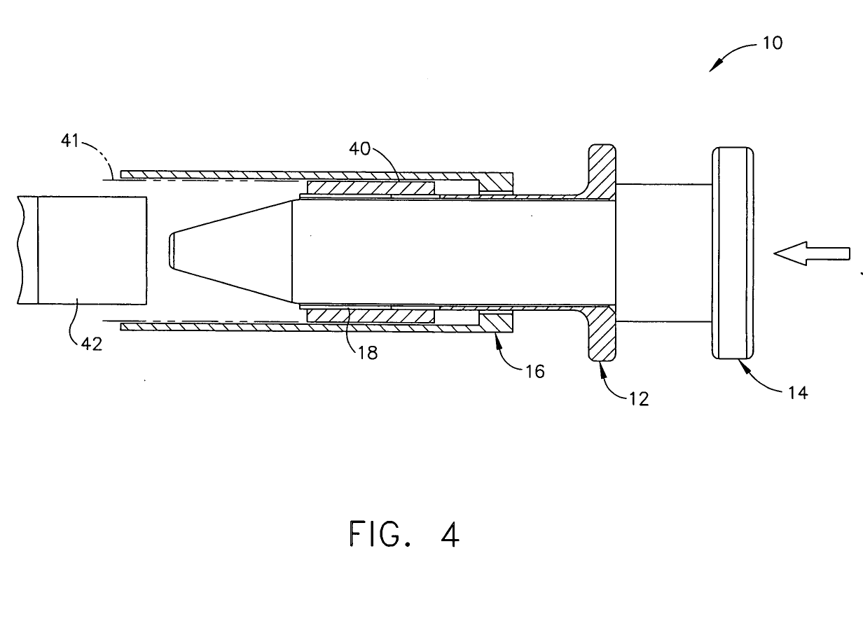

FIG. 4 shows plunger 14 forcefully extended through insertion end 18, thereby changing insertion end 18 from the collapsed to the expanded configuration, and expanding end cap 40. The user may hold handle 24 of temporary carrier 12 while pushing actuating element 32 (FIG. 2).

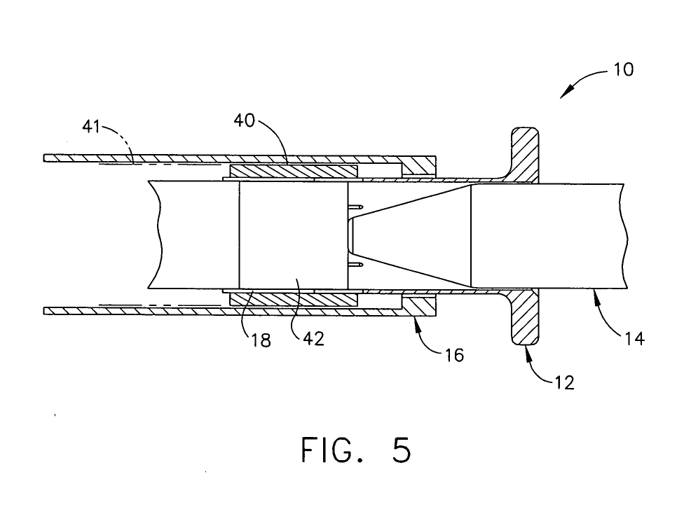

FIG. 5 shows plunger 14 partially withdrawn from temporary carrier 12 and endoscope tip 42 positioned inside insertion end 18 of temporary carrier 12 and end cap 40.

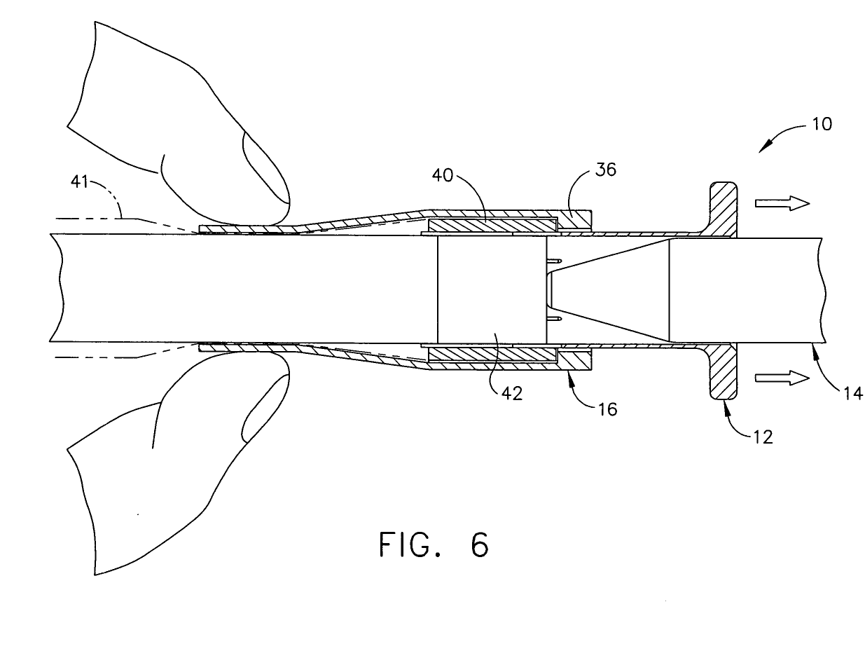

FIG. 6 shows temporary carrier 12 being separated from end cap 40. Ring 36 has been moved from a proximal position (as shown in FIGS. 2-5) on temporary carrier 12 to a distal position on insertion end 18 and adjacent to endcap 40. The user holds ring 36 against endcap 40 by gripping the opposing pair of extension elements 38 of retaining component 16 onto endoscope 42. In this manner, the user may maintain the position of end cap 40 relative to endoscope tip 42 while separating temporary carrier 12 from endcap 40.

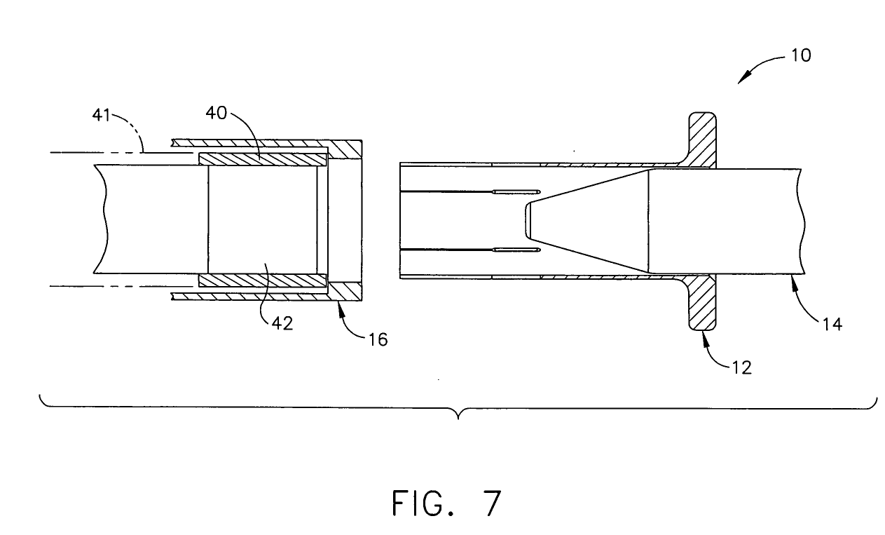

FIG. 7 shows temporary carrier 12 and plunger 14 separated from endcap 40 and endoscope 42, permitting end cap 40 to contract tightly around the periphery of endoscope tip 42.

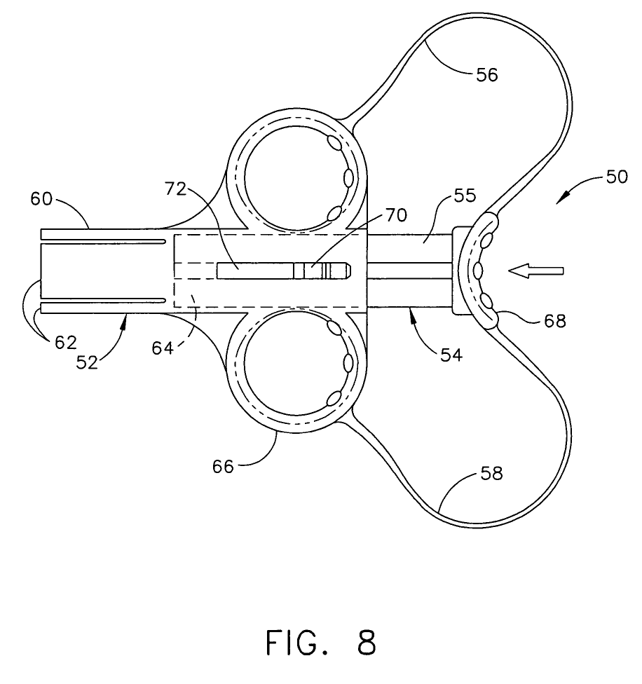

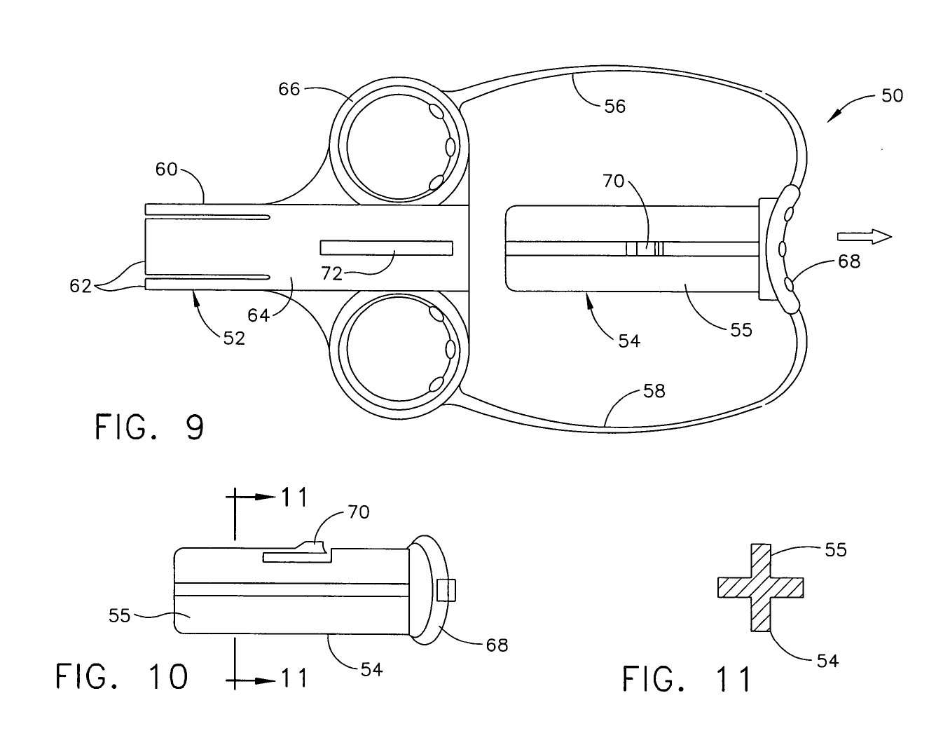

FIG. 8 is a front view of a second embodiment of an attachment tool 50 for attaching an endcap to an endoscope tip, showing a plunger 54 partially inserted into a temporary carrier 52 of attachment tool 50. FIG. 9 is the same view of attachment tool 50 as shown in FIG. 8, but showing plunger 54 withdrawn from, but still attached to, temporary carrier 52 by a pair of flexible connecting elements 56, 58. Attachment tool 50 also may include a retaining component (not shown) that is similar to retaining component 16 of FIG. 1

Temporary carrier 52 may include a body 64 having a bore (hidden) therethrough, wherein body 64 may include an insertion end 60 having a plurality of fingers 62. Temporary carrier 52 may further include a handle 66, which may be configured as a pair of finger grips. As may be seen more clearly in FIGS. 10 and 11, plunger 54 may have a plunger body 55 that has a cross-shaped, cross-sectional profile. An push-pull knob 68 disposed on the proximal end of plunger 54 may be thumb-actuated while two fingers are inserted into handle 66 so that attachment tool 50 may be operated much like a conventional syringe instrument.

Plunger 54 may further include a tab 70 attached to plunger body 55 for sliding engagement with a slot 72 formed in body 64 of temporary carrier 52. When plunger 54 is inserted into temporary carrier 52 as shown in FIG. 8, plunger 54 may be moved between a predetermined distal position (corresponding to the position of plunger 14 in FIG. 3) and a predetermined proximal position (corresponding to the position of plunger 14 in FIG. 4). Plunger 54 may be moved distally until tab 70 engages the distal end of slot 72. Similarly, plunger 54 may be moved proximally until tab 70 engages the proximal end of slot 72. An additional benefit, as compared to the first embodiment of attachment tool 10 shown in FIG. 1, of providing tab 70 and slot 72 is that a predetermined, maximal reception depth of endoscope tip 42 into temporary carrier 52 (see FIG. 5) may be provided when the user holds plunger 54 in the proximal position, thus eliminating the need for the user to estimate the maximal reception depth.

As shown in FIG. 10, tab 70 may be springably attached to or formed on plunger body 55 so that plunger 54 may be depressed and easily inserted into temporary carrier 52. Attachment tool 50 may be unitarily formed, such as by injection molding, into the configuration shown in FIG. 9. When plunger 54 is inserted into temporary carrier 52 as shown in FIG. 8, flexible connecting elements 56, 58 may apply a spring force to plunger 54 in the proximal direction so that plunger 54 moves to the proximal position when the user releases actuating element 68.

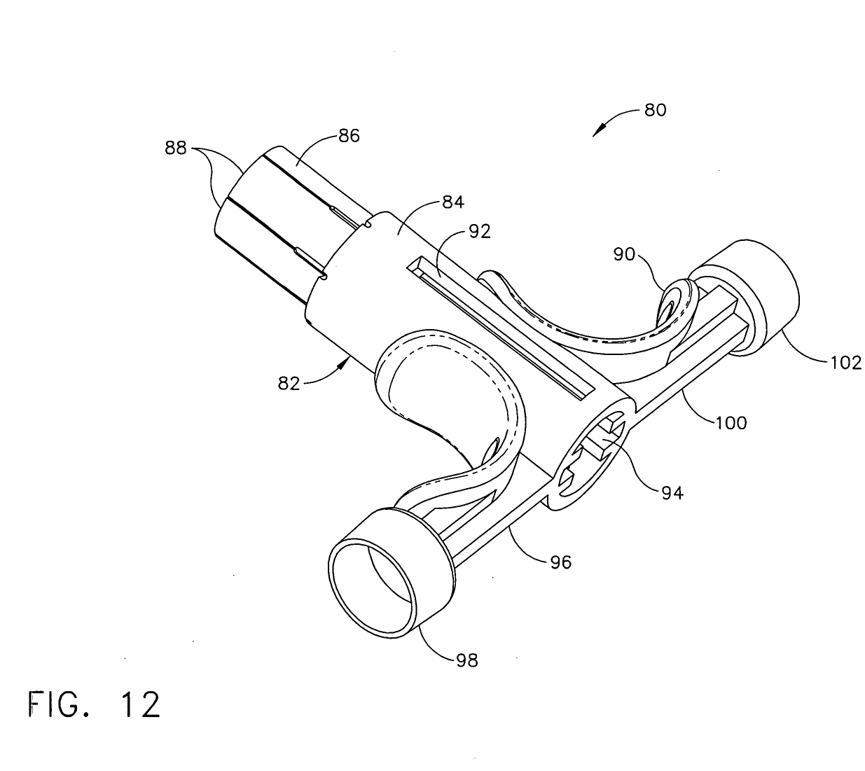

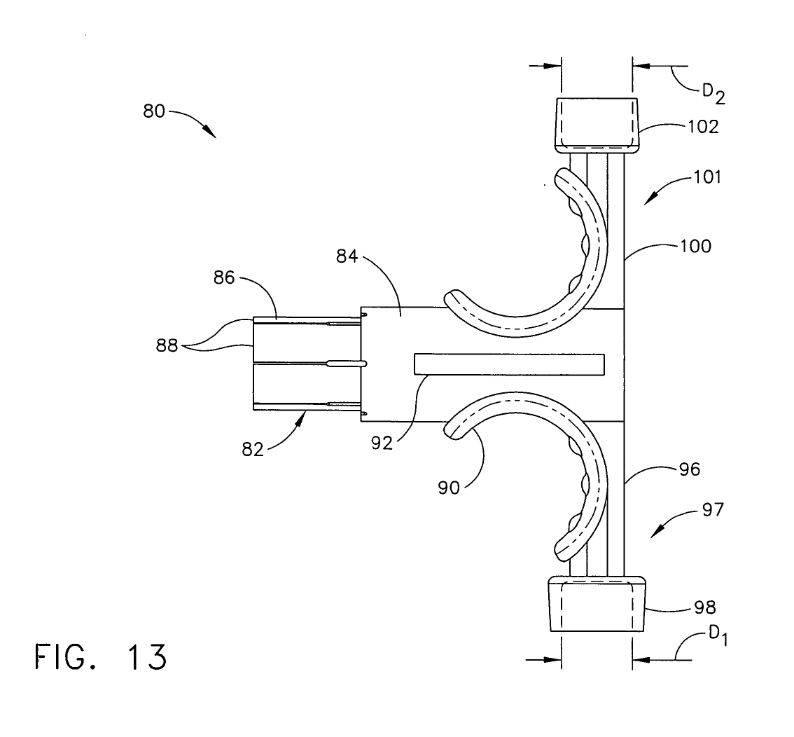

FIG. 12 is an isometric view and FIG. 13 is a top view of a temporary carrier 82 of a third embodiment of an attachment tool 80 for attaching an endcap to an endoscope. Attachment tool 80 may also include a retaining component and a plunger (each not shown) that are similar to what was described for attachment tool 50 of FIG. 8. Temporary carrier 82 may include a body 84 with an insertion end 86 having a plurality of fingers 88 as in the previous embodiments. Temporary carrier 82 may further include a handle 90, which in this embodiment is configured as a pair of semi-circular finger rests. Temporary carrier 82 may also include a slot 92 for operative engagement with a tab on the plunger, as described for attachment tool 50 of FIG. 8.

As may be seen in FIG. 12, the proximal portion of a bore 94 extending through body 84 has a cross-shaped, cross-sectional profile to match the cross-sectional profile of the plunger. Although not visible, the distal portion of bore 94 has a circular profile to receive the distal end of an endoscope.

Temporary carrier 82 is shown in FIG. 12 to also include a first and a second endcap detaching member 97, 101, respectively. (It is also possible for temporary carrier 82 to include only one or more than two endcap detaching members.) Detaching members 97, 101 may be used to facilitate removal of the endcap/apparatus from the endoscope to prepare the endoscope for the next medical procedure. First detaching member 97 may include an arm 96 extending outwardly from the proximal portion of body 84, and a cup 98 attached to the end of arm 96. Cup 98 may have an inside diameter (D1) that is sized to fit tightly to the tip of a first endoscope having a diameter, for example, of 12 millimeters. Similarly, second detaching member 101 may include an arm 100 extending outwardly opposite of arm 96, and a cup 102 having an inside diameter (D2) that is sized to fit tightly to the tip of a second endoscope having a diameter, for example, of 10 millimeters. The user may hold the endoscope and endcap in one hand and use the other hand to twist and push the appropriate cup, 98 or 102, in between the inside of the endcap and the periphery of the endoscope tip. Once the endcap is sufficiently positioned on the outside of the cup, the endoscope may be easily separated from the endcap.

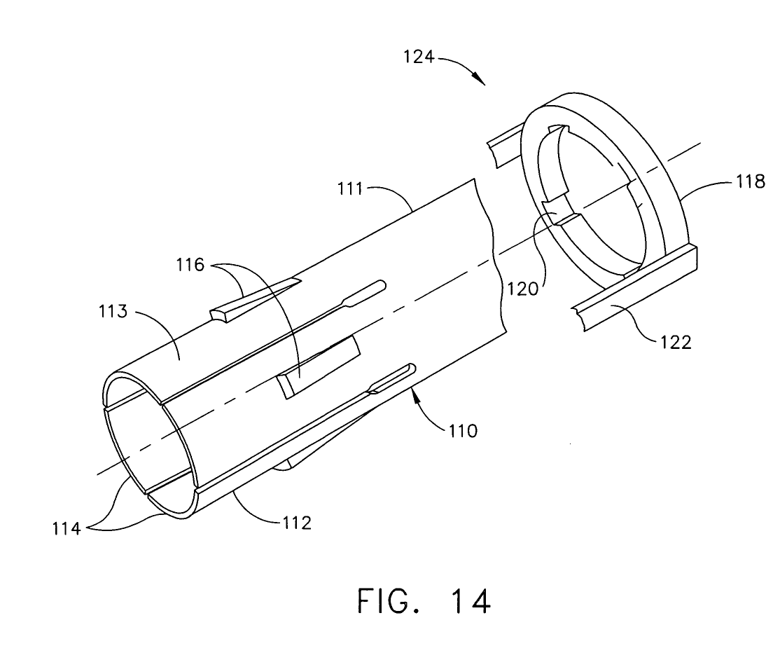

FIG. 14 is a partial, isometric view of an alternate version of the exchange and retaining components of the attachment tool embodiments described herein. A temporary carrier 110 may include a body 111 having an insertion end 112 that includes a plurality of fingers 114. Temporary carrier 110 may further include a plurality of endcap stops 116 peripherally arranged on a body surface 113 around body 111 as shown in FIG. 14, although it is also possible for temporary carrier 110 to include only one endcap stop 116. Endcap stops 116 may be configured as ramped keys as shown in FIG. 14 and may be unitarily formed with body 111. Endcap stops 116 predetermine a maximal insertion depth of insertion end 112 into the endcap (see FIG. 3) according to the longitudinal position of endcap stops 116 on insertion end 112, thereby eliminating the need for the user to estimate the insertion depth.

As shown in FIG. 14, retaining component 124 may include a ring 118 having a plurality of keyways 120 that may be aligned with endcap stops 116 so that ring 118 may be moved over endcap stops 116 and positioned adjacent to the endcap as shown in FIG. 6. A pair of extension elements 122 may be attached to ring 118 as described for retaining component 16 of FIG. 1 to hold ring 118 stationary while temporary carrier 110 is being separated from the endcap.

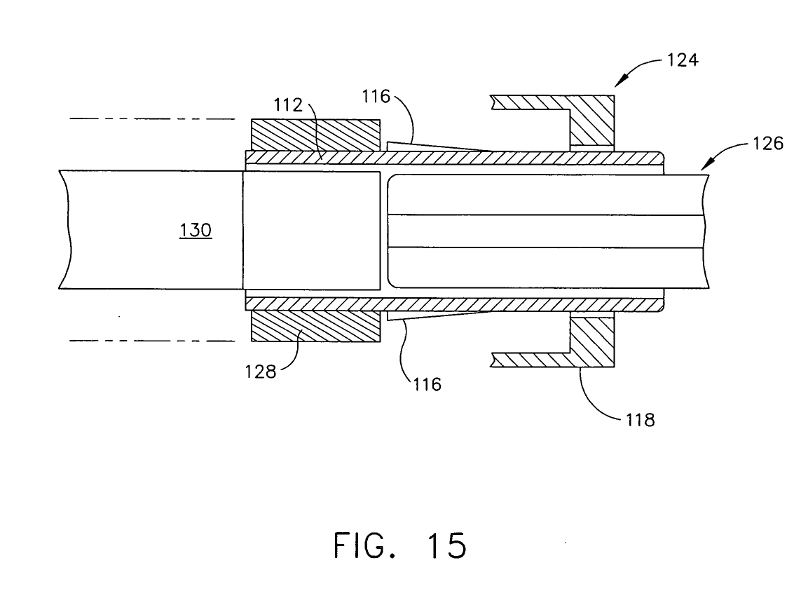

FIG. 15 is a longitudinal section of a portion of temporary carrier 110 and retaining component 124 shown in FIG. 14, and shown with a plunger 126 that may be similar to plunger 54 of FIG. 8. An end cap 128 is shown positioned over insertion end 112 of temporary carrier 110, and an endoscope 130 is shown positioned inside insertion end 112. Endcap 128 is shown abutting endcap stops 116, thereby correctly positioning insertion end 112 inside of endcap 128.

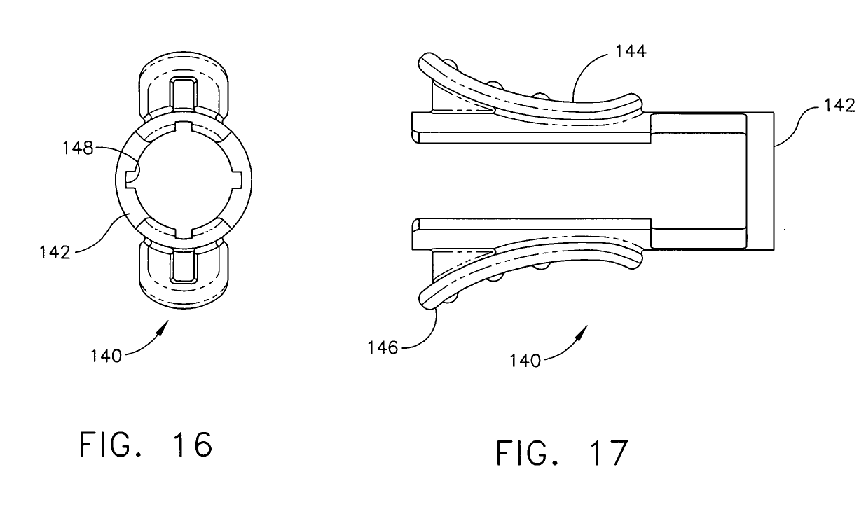

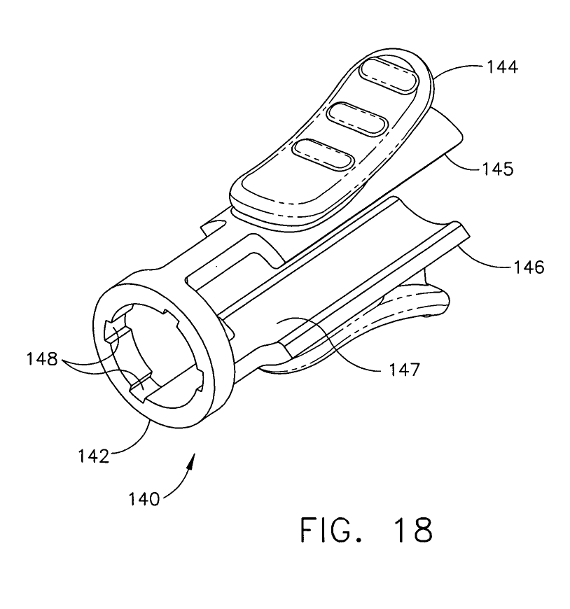

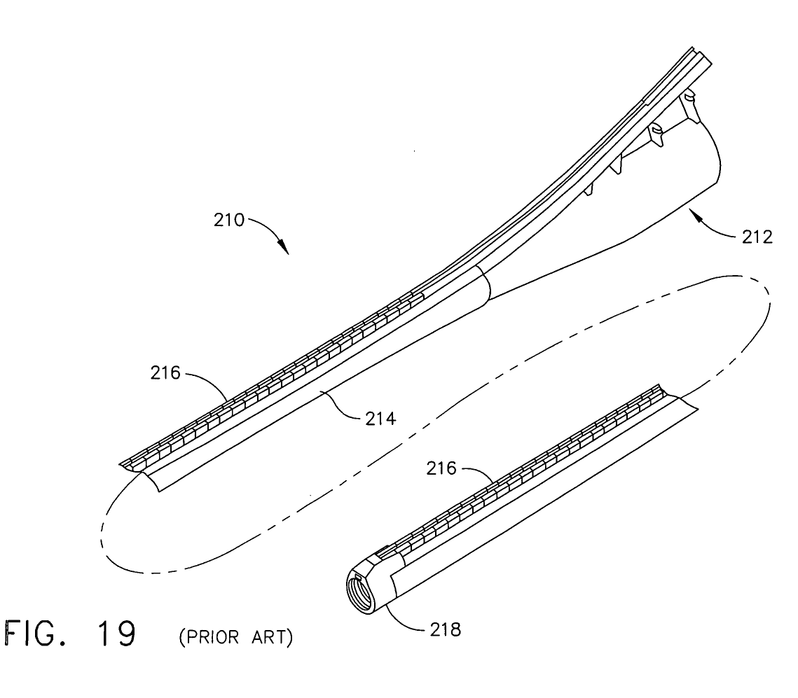

FIG. 16 is an end view, FIG. 17 is a side view and FIG. 18 is an isometric view of another alternate version of a retaining component 140. Retaining component 140 may include a pair of opposable finger grips 144, 146 springably attached to a ring 142 having a plurality of keyways 148 such as described for retaining component 124 of FIG. 14. Finger grip 146 may include a grip surface 147 and finger grip 144 may include a grip surface 145 (hidden), wherein each of surfaces 144, 147 are shaped to generally conform to the shape of the endoscope, thereby improving the user's ability to hold the endcap in the desired longitudinal position and angular orientation with respect to the endoscope tip while the temporary carrier is removed, as shown in FIG. 6.

Although an attachment tool and method of use has been shown and described with respect to certain aspects, variations, and embodiments, it should be understood that modifications may occur to those skilled in the art. What is claimed is:

1. A tool for attaching an endcap to the distal end of a tubular instrument, wherein the endcap includes an endcap opening having an unexpanded diameter that is smaller than the outer diameter of the instrument and an expanded diameter that is greater than the outer diameter of the instrument, the tool comprising:
a temporary carrier having a tubular body with a bore therethrough, wherein the body has a collapsible end that can assume a collapsed configuration and an expanded configuration, and wherein the collapsible end can be inserted into the endcap opening when the endcap opening has an unexpanded diameter and the collapsible end is in the collapsed configuration, and the collapsible end may receive the instrument when the collapsible end is in the expanded configuration and holding the endcap so that the endcap opening has the expanded diameter; and
a plunger that is slidingly insertable into the bore and extendable through the collapsible end, thereby expanding the end from the collapsed to the expanded configuration while holding the endcap thereon and expanding the endcap opening to the expanded diameter;
whereby the endcap may be positioned on the instrument while the endcap opening has the expanded diameter.

2. The tool of claim 1, wherein the temporary carrier retains the expanded configuration when the endcap is in place on the carrier and the plunger is withdrawn.

3. The tool of claim 2, wherein the tubular body is round and the collapsible end includes a plurality of circumferentially arranged fingers extending parallel to the bore axis.

4. The tool of claim 3, wherein the fingers support each other when a radially inward force is applied around the periphery of the collapsible end, so that the collapsible end maintains the expanded configuration.

5. The tool of claim 4, further comprising a retainer operatively associated with the temporary carrier and movable with respect to the temporary carrier, wherein the retainer holds the endcap at the desired position on the instrument when the support is withdrawn.

6. The tool of claim 5, wherein the retaining component includes a ring slidably retained on the temporary carrier, and at least one extension element attached to the ring and adapted to be held against the endoscope while the ring is positioned against the endcap.

7. The tool of claim 6, wherein the retaining component includes a pair of opposing extension elements configured as finger grips that may be pinched with the endoscope positioned therebetween to hold the ring against the endcap.

8. The tool of claim 1, wherein the instrument is an endoscope.

9. The tool of claim 8, wherein the insertion end of the temporary carrier when in the expanded configuration may receive the endoscope tip to a maximal reception depth as determined by the position of the plunger in the bore.

10. The tool of claim 9, wherein the body of the temporary carrier includes a slot extending parallel to the bore axis and operatively associated with a tab on the plunger, such that longitudinal position of the plunger in the bore may be limited to be between a predetermined proximal position and a predetermined distal position.

11. The tool of claim 1, further including a handle attached to the body of the temporary carrier.

12. The tool of claim 11, wherein the handle includes a pair of finger grips configured like a syringe grip and the plunger includes a push-pull knob.

13. The tool of claim 11, wherein the handle includes at least one cup sized to fit over the distal end of an endoscope, whereby the cup may be forcibly inserted between the periphery of the instrument tip and the endcap attached thereto in order to facilitate removal of the endcap from the endoscope.

14. The tool of claim 1, wherein the temporary carrier and the plunger are unitarily formed from a polymer.

15. The tool of claim 1, wherein the temporary carrier includes at least one endcap stop disposed on an outer surface of the body such that the insertion end may be inserted into the opening of the endcap to a maximal insertion depth when the endcap abuts the endcap stop, and wherein the maximal insertion depth is predetermined by the longitudinal position of the endcap stop on the body.

16. A method for attaching an endcap to the distal end of a medical instrument having an instrument tip, wherein the endcap includes an elastically deformable, endcap opening having an unexpanded diameter that is smaller than the diameter of the endoscope tip and an expanded diameter that is greater than the diameter of the instrument tip, the method comprising:
providing a tool comprising:
a first component adapted to hold the endcap and to receive the instrument tip; and
a second component operatively associated with the first component and movable with respect to the first component, wherein the second component is adapted to apply a force to the first component;
whereby the endcap opening may be elastically deformed to have the expanded diameter prior to positioning the endcap at a desired longitudinal location and angular orientation with respect to the instrument tip;
inserting the first component into the opening of the endcap;
applying a force to the first component by moving the second component with respect to the first component such that the opening of the endcap has the expanded diameter; and
positioning the endcap at a desired longitudinal location and angular orientation with respect to the endoscope tip.

17. The method of claim 16, further comprising:
holding the endcap at the desired longitudinal location and angular orientation; and
separating the first component from the endcap.

18. The method of claim 16, wherein the first component may hold the endcap independently of the second component while the endcap opening has the expanded diameter.

19. The method of claim 16, wherein the medical instrument is an endoscope.

20. The method of claim 19, wherein the first component is a temporary carrier having collapsed and expanded configurations and the second component is a plunger.
